(19) United States
(12) Patent Application Publication
(10) Pub. No.: US 2024/0290038 A1
(43) Pub. Date: Aug. 29, 2024

(54) GRID MODEL GENERATION METHODS AND SYSTEMS

(71) Applicant: SHANGHAI UNITED IMAGING HEALTHCARE CO., LTD., Shanghai (CN)

(72) Inventor: Jian GUO, Shanghai (CN)

(73) Assignee: SHANGHAI UNITED IMAGING HEALTHCARE CO., LTD., Shanghai (CN)

(21) Appl. No.: 18/589,373

(22) Filed: Feb. 27, 2024

(30) Foreign Application Priority Data
Feb. 27, 2023 (CN) 202310177324.1

(51) Int. Cl. G06T 17/20; G06T 7/50; G06T 19/20; G16H 30/40

(52) U.S. Cl. CPC G06T 17/20; G06T 7/50; G06T 19/20; G16H 30/40; G06T 2207/30101; G06T 2210/41; G06T 2219/2021

(57) ABSTRACT

The present disclosure provides a grid model generation method, device, and storage medium. The grid model generation method includes obtaining a three-dimensional contour point cloud related to an object; determining a plurality of two-dimensional (2D) contour point clouds based on the three-dimensional contour point cloud; generating a plurality of grid sections between two adjacent two-dimensional contour point clouds of the plurality of two-dimensional contour point clouds; and generating a grid model of the object based on the plurality of grid sections

### CROSS-REFERENCE TO RELATED APPLICATIONS

This application claims priority to Chinese Patent Application No. 202310177324.1, filed on Feb. 27, 2023, the entire contents of which are hereby incorporated by reference.

### TECHNICAL FIELD

The present disclosure relates to the field of grid model, and in particular, to a grid model generation method and system.

### BACKGROUND

A grid model is a model that approximates a three-dimensional (3D) object with a plurality of polygons (usually triangles) of similar sizes and shapes. The grid model is commonly used in various fields to generate virtual structures that simulate actual three-dimensional objects. For example, in the medical field, grid models can be used to create virtual organs that can be applied in various treatment scenarios, such as simulating a condition of an abnormity in a corresponding organ. However, existing algorithms for generating a grid model are complex and have low efficiency, which leads to a decrease in the efficiency of generating the virtual structures using the grid model.

Therefore, it is desirable to provide a grid model generation method and system to achieve a simple and neat process for generating the grid model, and improve the efficiency of generating a grid model, thereby effectively solving the problem that the algorithm for generating the grid model is relatively complex and the efficiency of generating the grid model is low.

### SUMMARY

One or more embodiments of the present disclosure provide a grid model generation method implemented on a machine including one or more processing devices and one or more storage devices. The grid model generation method may include obtaining a three-dimensional (3D) contour point cloud related to an object; determining a plurality of two-dimensional (2D) contour point clouds based on the three-dimensional contour point cloud; generating a plurality of grid sections between two adjacent two-dimensional contour point clouds of the plurality of two-dimensional contour point clouds; and generating a grid model of the object based on the plurality of grid sections.

In some embodiments, the plurality of grid sections may include a triangular section or a quadrangular section.

In some embodiments, the plurality of grid sections may include a triangular section. The generating a plurality of grid sections between two adjacent the two-dimensional contour point clouds of the plurality of two-dimensional contour point clouds may include determining, based on the two adjacent two-dimensional contour point clouds, a first two-dimensional contour point cloud and a second two-dimensional contour point cloud each of which includes a plurality of contour points; determining a plurality of first contour edges based on two adjacent contour points of the plurality of contour points in the first two-dimensional contour point cloud; for each of the plurality of first contour edges, determining a first contour point in the plurality of contour points of the second two-dimensional contour point cloud; generating a plurality of first triangular sections based on the first contour points and the first contour edges; determining a plurality of reconstructed contour points in the plurality of contour points of the second two-dimensional contour point cloud; determining a second contour edge based on two adjacent reconstructed contour points in the plurality of reconstructed contour points; determining a second contour point in the plurality of contour points of the first two-dimensional contour point cloud corresponding to the second contour edge; and generating a second triangular section based on the second contour point and the second contour edge.

In some embodiments, the first contour point may be a contour point in the second two-dimensional contour point cloud closest to the corresponding first contour edge. The second contour point may be a common vertex to two first triangular sections respectively including the two adjacent reconstructed contour points based on which the second contour edge is determined.

In some embodiments, a count of the plurality of contour points in the first two-dimensional contour point cloud may be greater than a count of the plurality of contour points in the second two-dimensional contour point cloud.

In some embodiments, the obtaining a three-dimensional (3D) contour point cloud related to an object may include obtaining a centerline of the object, wherein the centerline includes a plurality of center points; determining a reconstruction plane at each of the plurality of center points; generating the plurality of two-dimensional contour point clouds based on the reconstruction planes; and generating the three-dimensional contour point cloud based on the plurality of two-dimensional contour point clouds.

In some embodiments, the generating the plurality of two-dimensional contour point clouds based on the reconstruction planes may include for each of the plurality of two-dimensional contour point clouds, obtaining a contour expression and a contour rotation angle; generating the two-dimensional contour point cloud by sampling a preset count of contour points in the corresponding reconstruction plane based on the contour expression and the contour rotation angle.

In some embodiments, the determining a plurality of two-dimensional (2D) contour point clouds based on the three-dimensional contour point cloud may include obtaining a centerline of the object based on the three-dimensional contour point cloud, wherein the centerline includes a plurality of center points; determining a reconstruction plane at each of the plurality of center points; for each of the plurality of two-dimensional contour point clouds, determining the two-dimensional contour point cloud based on one or more contour points in the three-dimensional contour point cloud that have a distance from the reconstruction plane less than a threshold.

In some embodiments, the grid model may be configured to represent a virtual blood vessel.

In some embodiments, the grid model generation method may further include selecting at least one of the plurality of two-dimensional contour point clouds; and adjusting a contour expression of the at least one of the plurality of two-dimensional contour point clouds to simulate an abnormity of the virtual blood vessel.

One or more embodiments of the present disclosure further provide a grid model generation system. The grid model generation system may include at least one storage device including a set of instructions, and at least one processing device in communication with the at least one storage device. When executing the set of instructions, the at least one processing device may be directed to perform operations. The operations may include obtaining a three-dimensional (3D) contour point cloud related to an object; determining a plurality of two-dimensional (2D) contour point clouds based on the three-dimensional contour point cloud; generating a plurality of grid sections between two adjacent two-dimensional contour point clouds of the plurality of two-dimensional contour point clouds; and generating a grid model of the object based on the plurality of grid sections.

In some embodiments, the plurality of grid sections may include a triangular section or a quadrangular section.

In some embodiments, the plurality of grid sections may include a triangular section. The generating a plurality of grid sections between two adjacent the two-dimensional contour point clouds of the plurality of two-dimensional contour point clouds may include determining, based on the two adjacent two-dimensional contour point clouds, a first two-dimensional contour point cloud and a second two-dimensional contour point cloud each of which includes a plurality of contour points; determining a plurality of first contour edges based on two adjacent contour points of the plurality of contour points in the first two-dimensional contour point cloud; for each of the plurality of first contour edges, determining a first contour point in the plurality of contour points of the second two-dimensional contour point cloud; generating a plurality of first triangular sections based on the first contour points and the first contour edges; determining a plurality of reconstructed contour points in the plurality of contour points of the second two-dimensional contour point cloud; determining a second contour edge based on two adjacent reconstructed contour points in the plurality of reconstructed contour points; determining a second contour point in the plurality of contour points of the first two-dimensional contour point cloud corresponding to the second contour edge; and generating a second triangular section based on the second contour point and the second contour edge.

In some embodiments, the first contour point may be a contour point in the second two-dimensional contour point cloud closest to the corresponding first contour edge. The second contour point may be a common vertex to two first triangular sections respectively including the two adjacent reconstructed contour points based on which the second contour edge is determined.

In some embodiments, a count of the plurality of contour points in the first two-dimensional contour point cloud may be greater than a count of the plurality of contour points in the second two-dimensional contour point cloud.

In some embodiments, the obtaining a three-dimensional (3D) contour point cloud related to an object may include obtaining a centerline of the object, wherein the centerline includes a plurality of center points; determining a reconstruction plane at each of the plurality of center points; generating the plurality of two-dimensional contour point clouds based on the reconstruction planes; and generating the three-dimensional contour point cloud based on the plurality of two-dimensional contour point clouds.

In some embodiments, the generating the plurality of two-dimensional contour point clouds based on the reconstruction planes may include for each of the plurality of two-dimensional contour point clouds, obtaining a contour expression and a contour rotation angle; generating the two-dimensional contour point cloud by sampling a preset count of contour points in the corresponding reconstruction plane based on the contour expression and the contour rotation angle.

In some embodiments, the determining a plurality of two-dimensional (2D) contour point clouds based on the three-dimensional contour point cloud may include obtaining a centerline of the object based on the three-dimensional contour point cloud, wherein the centerline includes a plurality of center points; determining a reconstruction plane at each of the plurality of center points; for each of the plurality of two-dimensional contour point clouds, determining the two-dimensional contour point cloud based on one or more contour points in the three-dimensional contour point cloud that have a distance from the reconstruction plane less than a threshold.

In some embodiments, the grid model may be configured to represent a virtual blood vessel, and the grid model generation method may further include selecting at least one of the plurality of two-dimensional contour point clouds; and adjusting a contour expression of the at least one of the plurality of two-dimensional contour point clouds to simulate an abnormity of the virtual blood vessel.

One or more embodiments of the present disclosure further provide a non-transitory computer readable medium, comprising at least one set of instructions. When executed by one or more processors of a computing device, the at least one set of instructions causes the computing device to perform a method. The method may include obtaining a three-dimensional (3D) contour point cloud related to an object; determining a plurality of two-dimensional (2D) contour point clouds based on the three-dimensional contour point cloud; generating a plurality of grid sections between two adjacent two-dimensional contour point clouds of the plurality of two-dimensional contour point clouds; and generating a grid model of the object based on the plurality of grid sections.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a block diagram illustrating an exemplary hardware structure of a grid model generation system executing a grid model generation method according to some embodiments of the present disclosure;

FIG. 2 is a flowchart illustrating an exemplary process of a grid model generation method according to some embodiments of the present disclosure;

FIG. 3 is a flowchart illustrating an exemplary process for obtaining a three-dimensional contour point cloud according to some embodiments of the present disclosure;

FIG. 4 is a diagram illustrating an exemplary grid model of a tubular shape generated by a grid model generation method according to some embodiments of the present disclosure;

FIG. 5A is a flowchart illustrating an exemplary process for generating a grid section according to some embodiments of the present disclosure;

FIG. 5B is a schematic diagram illustrating generating a grid section by candidate contour points according to some embodiments of the present disclosure;

FIG. 6 is a flowchart illustrating an exemplary process for determining two-dimensional contour point clouds according to some embodiments of the present disclosure;

FIG. 7 is a flowchart illustrating an exemplary process for generating a grid model according to some other embodiments of the present disclosure;

FIG. 8 is a first schematic diagram illustrating generating a grid section using a random sample consensus algorithm according to some embodiments of the present disclosure;

FIG. 9 is a second schematic diagram illustrating generating a grid section using a random sample consensus algorithm according to some embodiments of the present disclosure;

FIG. 10 is a third schematic diagram illustrating generating a grid section using a random sample consensus algorithm according to some embodiments of the present disclosure;

FIG. 11 is a fourth schematic diagram illustrating generating a grid section using a random sample consensus algorithm according to some embodiments of the present disclosure;

FIG. 12 is a fifth schematic diagram illustrating generating a grid section using a random sample consensus algorithm according to some embodiments of the present disclosure; and

FIG. 13 is a diagram illustrating exemplary modules of a grid model generation system according to some embodiments of the present disclosure.

### DETAILED DESCRIPTION

To more clearly illustrate the technical solutions related to the embodiments of the present disclosure, a brief introduction of the drawings referred to the description of the embodiments is provided below. Obviously, the drawings described below are only some examples or embodiments of the present disclosure. Those having ordinary skills in the art, without further creative efforts, may apply the present disclosure to other similar scenarios according to these drawings. Unless obviously obtained from the context or the context illustrates otherwise, the same numeral in the drawings refers to the same structure or operation.

It should be understood that “system”, “device”, “unit” and/or “module” as used herein is a manner used to distinguish different components, elements, parts, sections, or assemblies at different levels. However, if other words serve the same purpose, the words may be replaced by other expressions.

As shown in the present disclosure and claims, the words “one”, “a”, “a kind” and/or “the” are not especially singular but may include the plural unless the context expressly suggests otherwise. In general, the terms “comprise,”“comprises,”“comprising,”“include,”“includes,” and/or “including,” merely prompt to include operations and elements that have been clearly identified, and these operations and elements do not constitute an exclusive listing. The methods or devices may also include other operations or elements.

The flowcharts used in the present disclosure illustrate operations that systems implement according to some embodiments of the present disclosure. It should be understood that the previous or subsequent operations may not be accurately implemented in order.

Instead, each step may be processed in reverse order or simultaneously. Meanwhile, other operations may also be added to these processes, or a certain step or several steps may be removed from these processes.

Unless otherwise defined, technical terms or scientific terms referred to in the present disclosure shall have the ordinary meanings understood by a person of ordinary skill in the technical field to which the present disclosure belongs. In the present disclosure, the words “one”, “a”, “a kind”, “the”, and “these”, etc., are not quantitative limitations but may be singular or plural. The terms “comprise,”“comprises,”“comprising,”“include,”“includes,”“including,”“have,”“has,” and/or “having” and any variations thereof, are intended to cover non-exclusive inclusion. For example, processes, methods and systems, products, or devices that include a series of operations or modules (units) are not limited to the listed operations or modules (units), but may include operations or modules (units) that are not listed, or may include other operations or modules (units) inherent in such processes, methods, products, or devices.

As used in the present disclosure, the terms “connect”, “attach”, “couple”, etc., used in the present disclosure are not limited to physical or mechanical connections, but may include electrical connections, whether connections are direct or indirect. As used in the present disclosure, “a plurality of” refers to two or more. The term “and/or” describes an associative relationship of the related objects, indicating that three relationships may exist, e.g., “A and/or B” may indicate that only A exists, both A and B exist, and only B exists. Usually, the character “/” indicates that the related objects are in an “or” relationship. In the present disclosure, the terms “first”, “second”, “third”, etc. are used in the present disclosure only to distinguish between similar objects and do not represent a specific ordering of the objects.

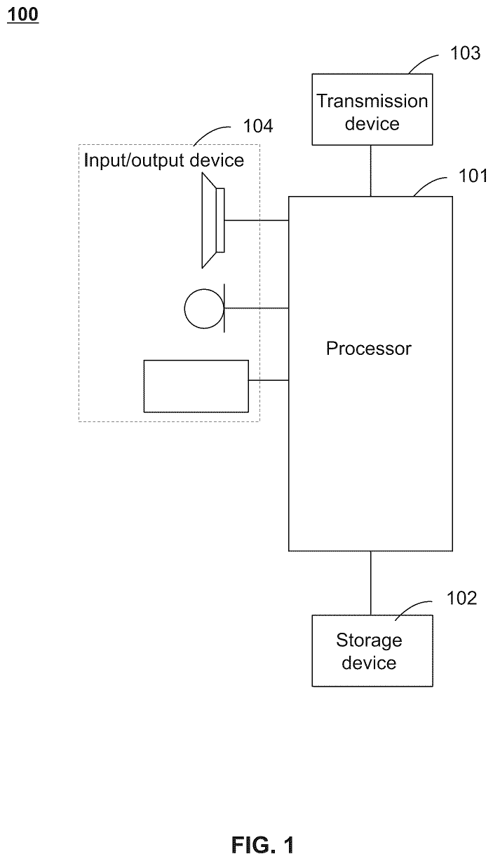

FIG. 1 is a block diagram illustrating an exemplary hardware structure of a grid model generation system executing a grid model generation method according to some embodiments of the present disclosure.

The grid model generation system may be implemented on a terminal, a processing device, or other similar computing devices. The terminal may include a mobile device, a tablet computer, a laptop computer, or the like, or any combination thereof. The processing device may be a single server, or a server group. The server group may be centralized or distributed.

In some embodiments, the processing device may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, a grid model generation system 100 may include at least one storage device storing computer instructions, and at least one processing device configured to execute at least part of the instructions to implement the grid model generation method described in any of the embodiments of the present disclosure.

As shown in FIG. 1, the grid model generation system 100 may include at least one processor 101 and at least one storage device 102 (only one of which is shown in FIG. 1), and the storage device 102 may be configured to store data.

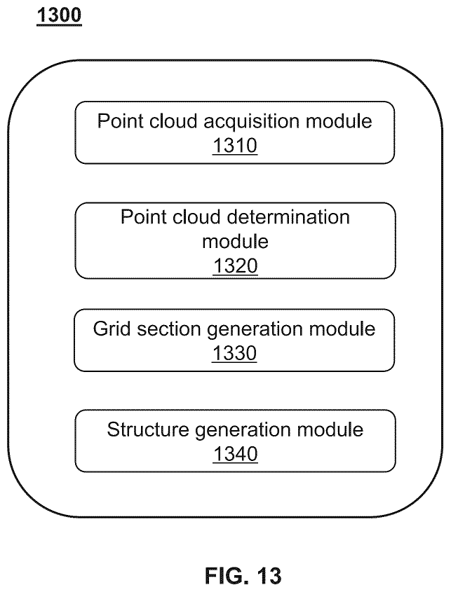

In some embodiments, the processor 101 may obtain data and/or information obtained from other devices/assemblies or components. The processor 101 may execute program instructions based on data, information, and/or processing results to perform one or more functions described in embodiments of the present disclosure. Merely by way of example, the processor 101 may include but is not limited to, a central processing unit (CPU), a microcontroller unit (MCU), an application-specific integrated circuit (ASIC), a field-programmable gate array (FPGA), etc., or any combinations thereof. In some embodiments, the processor 101 may include a plurality of modules, such as a point cloud acquisition module, a point cloud determination module, a grid section generation module, and a structure generation module, etc. Different modules may be configured to separately execute different program instructions, which may be described in detail in FIG. 13 and related descriptions thereof.

The storage device 102 may be configured to store computer instructions or computer programs. For example, software programs and modules of application software, such as a computer program corresponding to the grid model generation method in the embodiments of the present disclosure. The processor 101 may perform various functional applications and data processing by executing the computer instructions or the computer programs stored in the storage device 102, i.e., the grid model generation method described in the embodiment of the present disclosure may be implemented. The storage device 102 may include a high-speed random access memory and a non-volatile memory, such as one or more magnetic storage devices, a flash memory, or other non-volatile solid-state memory. In some examples, the storage device 102 may further include memories set remotely relative to the processor 101, and the remote memories may be connected to a terminal via a network. The network may include but is not limited to, the Internet, an enterprise intranet, a local area network, a mobile communication network, and any combination thereof.

In some embodiments, the grid model generation system 100 may further include a transmission device 103 and an input/output device 104. The transmission device 103 and the input/output device 104 may be connected with the processor 101, respectively. Both the transmission device 103 and the input/output device 104 may be configured to perform communication functions. It should be understood that the structure shown in FIG. 1 is merely illustrative, and does not limit the structure of the grid model generation system 100. For example, the grid model generation system 100 may also include more or fewer components than those shown in FIG. 1, or have a different configuration than that shown in FIG. 1.

The transmission device 103 may be configured to receive or send data via the network. The network may include a wireless network provided by a communication provider. In some embodiments, the transmission device 103 may include a network interface controller (NIC) that may be connected with other network devices via a base station to communicate with the

Internet. In some embodiments, the transmission device 103 may be a radio frequency (RF) module for communicating with the Internet wirelessly.

The input/output device 104 may be a device that interacts with a user. The input/output device 104 may be a device that interacts with the user via voice, text, images, or the like. For example, the input/output device 104 may include an interaction interface, a sound interaction device, or the like.

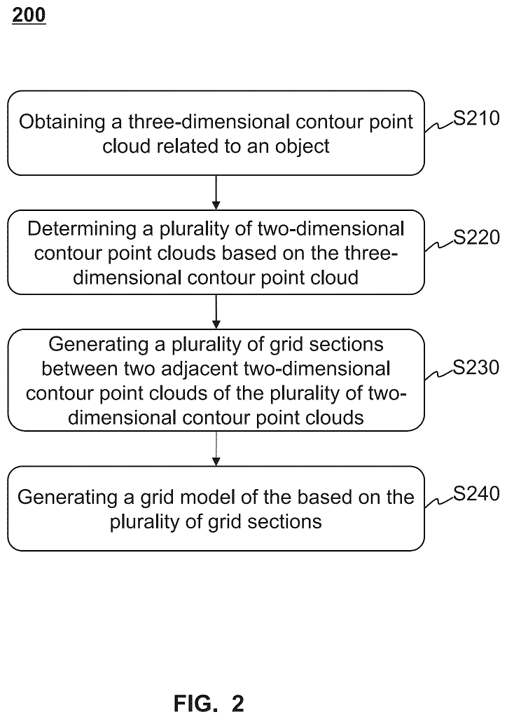

FIG. 2 is a flowchart illustrating an exemplary process of a grid model generation method according to some embodiments of the present disclosure. In some embodiments, the process 200 may be implemented in the grid model generation system 100 illustrated in FIG. 1. For example, the process 200 may be stored in a storage medium (e.g., the storage device 102) as a form of instructions, and can be invoked and/or executed by the grid model generation system 100 (e.g., the processor 101, or one or more modules illustrated in FIG. 13). The operations of the illustrated process 200 presented below are intended to be illustrative. In some embodiments, the process 200 may be accomplished with one or more additional operations not described, and/or without one or more of the operations discussed. Additionally, the order in which the operations of the process 200 as illustrated in FIG. 2 and described below is not intended to be limiting.

In S210, the grid model generation system 100 may obtain a three-dimensional (3D) contour point cloud related to an object.

A grid model refers to a model that uses a plurality of polygons (e.g., triangles, quadrangles, etc.) to represent a three-dimensional (3D) contour of a 3D object. A virtual structure may be generated based on a grid model, and the virtual structure may effectively simulate a corresponding actual three-dimensional object. Exemplarily, in the medical field, various portions of a human body or organs, such as blood vessels, the heart, human limbs, a trachea, an intestine, or the like, may be simulated through different grid models. In the field of daily life, various household items, such as a water cup, a cell phone, or the like, may also be simulated through grid models. As used in the present disclosure, the object refers to an actual 3D object. The actual 3D object may be simulated by generating a grid model representing a virtual structure of the actual 3D object.

The three-dimensional contour point cloud may include a plurality of contour points. The plurality of contour points may represent the 3D external contour of the three-dimensional object. The three-dimensional contour point cloud may be a basis for reconstructing a grid model. Each contour point in the plurality of contour points may correspond to a three-dimensional coordinate.

In some embodiments, the processor 101 may directly obtain the three-dimensional contour point cloud from an external device. In some embodiments, the processor 101 may obtain the three-dimensional contour point cloud via a sensor. For example, the sensor may emit laser pulses to scan the 3D external contour of a three-dimensional object. The laser pulses may be reflected by the surface of the three-dimensional object and return to the sensor.

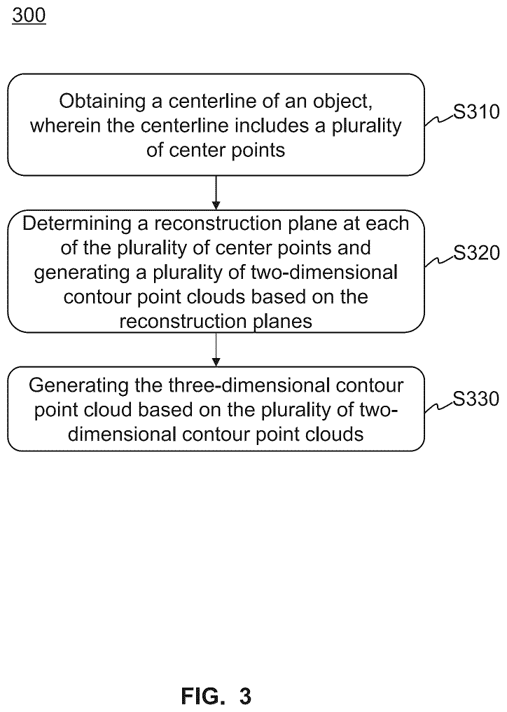

The sensor may generate point cloud data for representing the contour of the three-dimensional object, i.e., the three-dimensional contour point cloud, based on one or more features (e.g., a period from a time when a laser pulse is emitted to a time when the laser pulse is reflected back, an intensity of the reflected laser pulse, etc.) of returned laser pulses. In some embodiments, the processor 101 may obtain a three-dimensional model of a three-dimensional object and sample a plurality of sample points on a surface of the three-dimensional model. The plurality of sample points may form the three-dimensional contour point cloud. In some embodiments, in the medical field, the processor 101 may obtain medical image data of a three-dimensional object (e.g., various portions of the human body or the organs) and obtain the three-dimensional contour point cloud from the medical image data. For example, the processor 101 may obtain a three-dimensional medical image of a three-dimensional object and sample a plurality of sample points on the contour of the three-dimensional object based on the three-dimensional image to obtain the three-dimensional contour point cloud. As another example, the processor 101 may obtain a plurality of two-dimensional (2D) images each of which corresponds to a slice of the three-dimensional object. For each two-dimensional image in the plurality of two-dimensional images, a plurality of sample points may be sampled on a contour of the corresponding slice based on the 2D image. The sample points of the plurality of two-dimensional images may form the three-dimensional contour point cloud. In some embodiments, the three-dimensional contour point cloud may be generated based on a preset point cloud generation algorithm (e.g., a three-dimensional contour point cloud generation process as described in FIG. 3) by a processing device (e.g., the processor 101). The processor 101 may obtain a centerline of an object, and the centerline may include a plurality of center points. The processor 101 may determine a reconstruction plane at each of the plurality of center points, generate a plurality of two-dimensional contour point clouds based on the reconstruction planes, and generate the three-dimensional contour point cloud based on the plurality of two-dimensional contour point clouds. More descriptions regarding obtaining the three-dimensional contour point cloud may be found in FIG. 3 and related descriptions thereof, which may not be repeated here.

In S220, the grid model generation system 100 may determine a plurality of two-dimensional contour point clouds based on the three-dimensional contour point cloud.

After obtaining the three-dimensional contour point cloud, the processor 101 may analyze a structure of the three-dimensional contour point cloud. In some embodiments, the processor 101 may extract a plurality of layers of two-dimensional contour point clouds stacked together from the three-dimensional contour point cloud. The two-dimensional contour point cloud may include at least two contour points in the three-dimensional contour point cloud.

The at least two contour points of the two-dimensional contour point cloud may be in the same layer structure. The layer structure may be a plane or a non-plane (e.g., a curved plane, a folded plane, etc.). The plurality of contour points in the two-dimensional contour point cloud may be typically distributed in a circular pattern.

In some embodiments, the processor 101 may divide the three-dimensional contour point cloud into a plurality of layer structures stacked together, and determine a two-dimensional contour point cloud based on the contour points in each layer structure.

In some embodiments, the processor 101 may determine the plurality of two-dimensional contour point clouds based on a count of contour points in each layer structure.

For each layer structure, in response to determining that a count of contour points in the layer structure is two or more, the processor 101 may determine the contour points in the layer structure as a two-dimensional contour point cloud. In response to determining that a count of contour points in the layer structure is one, the contour point may be excluded and may not be involved in grid model reconstruction. In other words, the processor 101 may exclude a layer structure that includes only one contour point and designate a layer structure including a plurality of contour points as a two-dimensional contour point cloud.

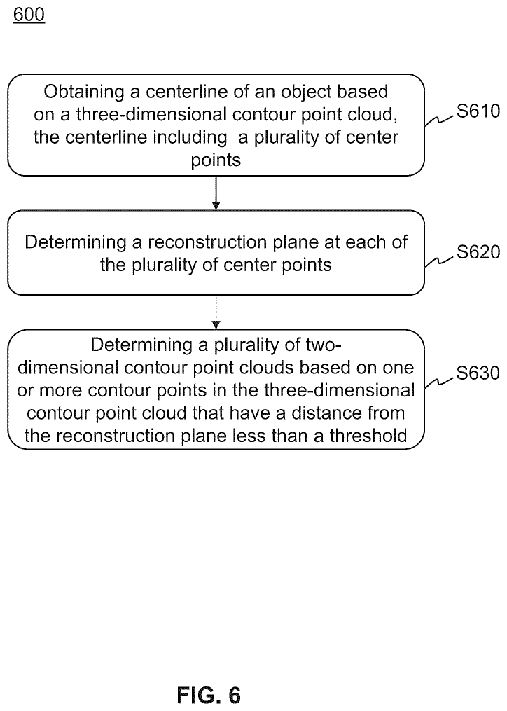

In some embodiments, the processor 101 may obtain the centerline of the object based on the three-dimensional contour point cloud. The centerline may include a plurality of center points. The processor 101 may determine a reconstruction plane at each of the plurality of center point. For each of the plurality of two-dimensional contour point clouds, the processor 101 may determine one or more contour points in the three-dimensional contour point cloud that have a distance from a corresponding reconstruction plane less than a threshold. The one or more contour points may be used to determine a two-dimensional contour point cloud. For example, in response to determining that a count of the one or more contour points is two or more, the processor 101 may determine the contour points as a two-dimensional contour point cloud. In response to determining that a count of the one or more contour points is one, the contour point may be excluded and may not be involved in grid model reconstruction. More descriptions may be found in FIG. 6 and related descriptions thereof.

It should be noted that, for a three-dimensional contour point cloud obtained directly from an external device, a three-dimensional contour point cloud obtained via the sensor, a three-dimensional contour point cloud obtained via the three-dimensional modeling model, and a three-dimensional contour point cloud obtained via the medical imaging data, the plurality of two-dimensional contour point clouds of a plurality of layers stacked sequentially in the three-dimensional contour point cloud may be determined by performing the above-described process (e.g., the process illustrated in FIG. 6). For a three-dimensional contour point cloud generated by a processing device using a preset point cloud generation algorithm (e.g., a three-dimensional contour point cloud generation process as described in FIG. 3), the three-dimensional contour point cloud may directly include information of the plurality of two-dimensional contour point clouds of a plurality of layers that are stacked in sequence. Therefore, in such a case, it is relatively simple to determine the plurality of two-dimensional contour point clouds in the three-dimensional contour point cloud.

In S230, the grid model generation system 100 may generate a plurality of grid sections between two adjacent two-dimensional contour point clouds based on contour points of the two adjacent two-dimensional contour point clouds.

In some embodiments, the processor 101 may generate the plurality of grid sections between two adjacent two-dimensional contour point clouds.

The plurality of grid sections refer to data structures used for generating a grid model and may be closed surfaces formed by a plurality of edges. The grid section may be a polygon and have at least three vertices. Contour points in the two-dimensional contour point clouds may be designated as the vertices of the polygons that form the plurality of grid sections.

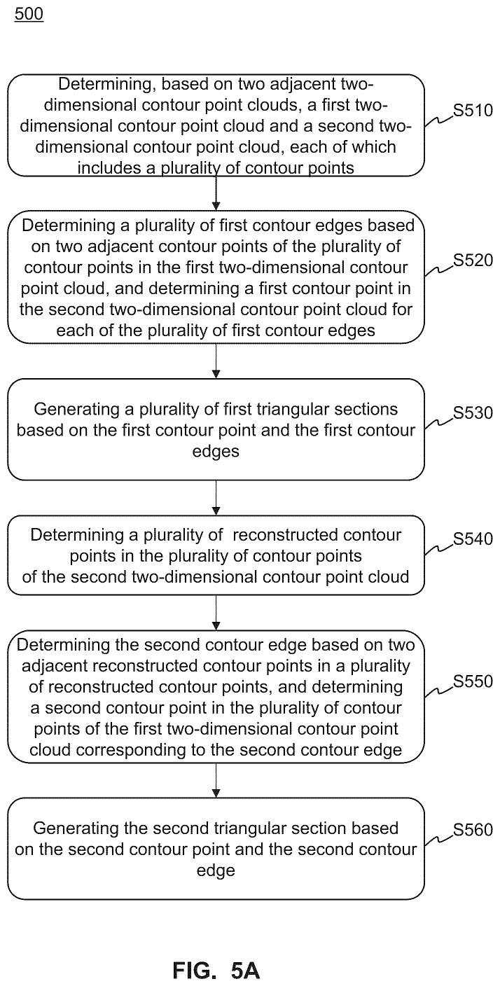

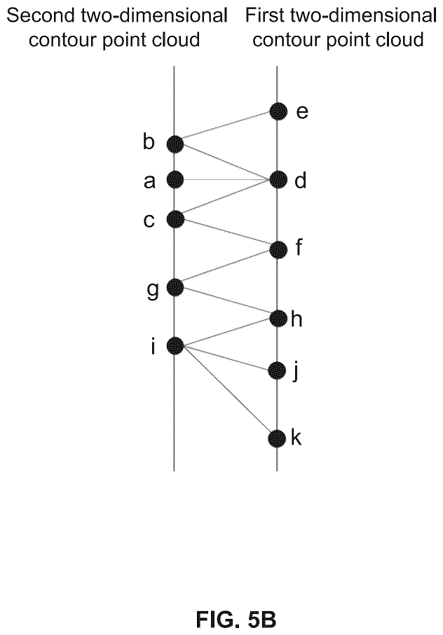

In some embodiments, the plurality of grid sections may include a triangular section and/or a quadrangular section. In three non-collinear contour points, a closed figure formed by connecting each two contour points is referred to as the triangular section. In four contour points of which any three contour points are also non-collinear, two adjacent contour points may be connected to form four non-collinear line segments, with endpoints of each of the four non-collinear line segments being connected, a closed figure formed by connecting two adjacent contour points is referred as the quadrilateral section. In some embodiments, when the plurality of grid sections include a triangular section, the processor 101 may select two contour points in one of two adjacent two-dimensional contour point clouds and one contour point in another of the two adjacent two-dimensional contour point clouds, and connect the three contour points to form a triangular section. In some embodiments, when the plurality of grid sections include a quadrangular section, the processor 101 may select two contour points in each of the two adjacent two-dimensional contour point clouds, respectively, and connect adjacent contour points of the four contour points to form a quadrangular section. In some embodiments, a plurality of grid sections may be formed between two adjacent two-dimensional contour point clouds because each two-dimensional contour point cloud includes a plurality of contour points. More descriptions regarding generating the plurality of grid sections may be found in FIG. 5 and related descriptions thereof, which may not be repeated here.

In some embodiments, for a complete three-dimensional contour point cloud, the generation of grid sections may be performed layer-by-layer. Merely by way of example, firstly, a plurality of grid sections may be generated between a first two-dimensional contour point cloud and a second two-dimensional contour point cloud that are adjacent, and then a plurality of grid sections may be generated between the second two-dimensional contour point cloud and a third two-dimensional contour point cloud that are adjacent. Since the generation of grid sections between two adjacent two-dimensional contour point clouds are related only to the two adjacent two-dimensional contour point clouds, and is independent of other two-dimensional contour point clouds, the generation of grid sections between any two adjacent two-dimensional contour point clouds may be performed simultaneously, which greatly improves efficiency of grid model generation.

It should be noted that not all two-dimensional contour point clouds in the three-dimensional contour point cloud need to participate in the generation of grid sections. Therefore, as used herein, two adjacent two-dimensional contour point clouds refer to two adjacent two-dimensional contour point clouds involved in the generation of grid sections. For a complete three-dimensional contour point cloud, cross-layer processing may be performed. For example, for a three-dimensional contour point cloud including ten two-dimensional contour point clouds, odd-numbered two-dimensional contour point clouds may be involved in the generation of grid sections, and even-numbered two-dimensional contour point clouds may not be involved in the generation of grid sections. The first two-dimensional contour point cloud and the third two-dimensional contour point cloud may be regarded as two adjacent two-dimensional contour point clouds, and the third two-dimensional contour point cloud and the fifth two-dimensional contour point cloud may be regarded as two adjacent two-dimensional contour point clouds, and so on.

In some embodiments, the cross-layer processing may be performed when the three-dimensional object has a relatively simple structure (e.g., a uniform tubular structure). In some embodiments, the cross-layer processing may be performed on a portion of the three-dimensional object that has a relatively simple and uniform structure.

In S240, the grid model generation system 100 may generate a grid model of the object based on the plurality of grid sections.

In some embodiments, when the plurality of grid sections are generated, a grid model may be generated based on the plurality of grid sections. Merely by way of example, the processor 101 may integrate the grid sections to generate a complete grid model. As another example, the processor 101 may further render the grid model after integrating the grid sections, then a complete virtual structure may be formed.

In some embodiments, the grid model may be configured to represent a virtual blood vessel. The processor 101 may select one or more two-dimensional contour point clouds and adjust a contour expression of the selected one or more two-dimensional contour point clouds to simulate an abnormity of the virtual blood vessel. For example, the processor 101 may first determine one or more 2D contour point clouds that are used to simulate an abnormal narrowing of the virtual blood vessel, and then adjust the contour expression of the one or more 2D contour point clouds to reduce the size of the one or more 2D contour point clouds, which causes a portion of the virtual blood vessel to become narrowed to simulate the abnormal narrowing of the virtual blood vessel.

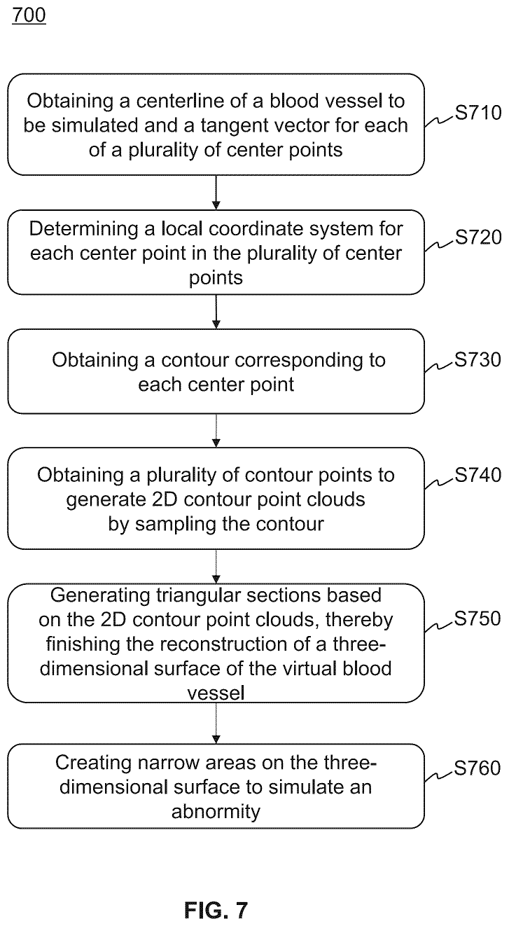

More descriptions regarding the contour expression and simulation of the virtual vessel may be found in FIG. 3 and FIG. 7 and related descriptions thereof.

In the embodiments of the present disclosure, a plurality of grid sections may be generated by obtaining a three-dimensional contour point cloud of an object. A plurality of two-dimensional contour point clouds stacked sequentially may be determined in the three-dimensional contour point cloud, and a plurality of grid sections between two adjacent two-dimensional contour point clouds may be generated. Since the plurality of grid sections may be generated between two adjacent two-dimensional contour point clouds, the generation of grid sections may be simple and neat, and the plurality of grid sections between any two adjacent two-dimensional contour point clouds may also be generated at the same time, which greatly improves efficiency of generating the grid model and solves a problem of complex grid model generation algorithms and low efficiency of grid model generation in related technologies.

It should be noted that the above description of the process 200 is intended to be exemplary and illustrative only, and does not limit the application scope of the present disclosure. For those skilled in the art, various corrections and changes may be made to the process 200 under the guidance of the present disclosure. However, these corrections and changes remain within the scope of the present disclosure.

FIG. 3 is a flowchart illustrating an exemplary process for obtaining a three-dimensional contour point cloud according to some embodiments of the present disclosure. In some embodiments, the process 300 may be implemented in the grid model generation system 100 illustrated in FIG. 1. For example, the process 300 may be stored in a storage medium (e.g., the storage device 102) as a form of instructions, and can be invoked and/or executed by the grid model generation system 100 (e.g., the processor 101, or one or more modules illustrated in FIG. 13). The operations of the illustrated process 300 presented below are intended to be illustrative. In some embodiments, the process 300 may be accomplished with one or more additional operations not described, and/or without one or more of the operations discussed. Additionally, the order in which the operations of the process 300 as illustrated in FIG. 3 and described below is not intended to be limiting.

In S310, the grid model generation system 100 may obtain a centerline of an object, wherein the centerline may include a plurality of center points.

The centerline of the object refers to a geometric centerline of the three-dimensional object. Merely by way of example, if the three-dimensional object is a tubular structure, the centerline of the three-dimensional object may be a geometric centerline along an extension direction of the tubular structure.

In some embodiments, the processor 101 may directly obtain the centerline from an external device or may generate the centerline by inputting a plurality of center points by a user. In some embodiments, for the centerline obtained directly from the external device, the processor 101 may analyze the obtained centerline to determine a plurality of center points of the centerline. In some embodiments, the processor 101 may also directly obtain a plurality of center points, and then generate the centerline based on the plurality of center points. Merely by way of example, the processor 101 may obtain at least one center point input by a user, and then perform an interpolation process on the at least one center point to obtain a plurality of center points distributing with a suitable density.

Exemplary interpolation processes may include B-spline interpolation, bilinear interpolation, or the like.

In some embodiments, the processor 101 may determine a count of the plurality of center points based on the complexity of the overall structure of the 3D object. For example, if the overall structure of the object is relatively complex, the count of center points may be relatively large. If the overall structure of the object is relatively simple, the count of center points may be relatively small.

In some embodiments, a distribution of the center points may be uniform (distributing at equal distances) or non-uniform. For example, for a relatively complex portion of the object, the distribution of the center points may be relatively dense. For a relatively simple portion of the object, the distribution of the center points may be relatively sparse.

In the embodiments of the present disclosure, the plurality of center points may be designed based on the complexity, allowing contour points to be closer to the structure of the object, which makes the generated grid model more accurate.

In S320, the grid model generation system 100 may determine a reconstruction plane at each of the plurality of center points and generate the plurality of two-dimensional contour point clouds based on the reconstruction planes.

The reconstruction plane refers to a plane that is reconstructed at a center point. In some embodiments, the processor 101 may determine a reconstruction plane at each of the plurality of center points, or each of a portion of the plurality of center points. The processor 101 may generate the plurality of two-dimensional contour point clouds in the reconstruction planes. The reconstruction planes may be constructed based on the plurality of center points, and a plurality of reconstruction planes may be discretely stacked, causing the plurality of the two-dimensional contour point clouds to also be discretely stacked.

In some embodiments, the processor 101 may determine a reconstruction plane by determining a tangent vector of the centerline at a center point, and then reconstruct a plane that passes through the center point and is perpendicular to the tangent vector. In some embodiments, the processor 101 may obtain an analytical expression of the centerline based on an interpolation algorithm, and determine tangent vectors of the centerline at different center points based on the analytical expression.

In some embodiments, generating the plurality of two-dimensional contour point clouds in the reconstruction planes may specifically include the following operations. For each of the plurality of two-dimensional contour point clouds, a contour expression and a contour rotation angle may be obtained, and the two-dimensional contour point cloud may be obtained by sampling a preset count of contour points based on the contour expression and the contour rotation angle.

The contour expression refers to a mathematical expression, such as functions, configured to describe a two-dimensional closed or unclosed contour. The contour rotation angle refers to a preset rotation angle of the contour expression around a center point corresponding to the reconstruction plane. When generating a two-dimensional contour point cloud in the reconstruction plane, the processor 101 may first determine the contour expression and the contour rotation angle for the two-dimensional contour point cloud. In some embodiments, for a pipe model, the contour expression may be an elliptic equation x²/a²+y²/b²=1. In some embodiments, the processor 101 may determine the contour expression and the contour rotation angle based on a randomized algorithm, or the like. For example, parameters a and b in the elliptic equation may be randomly determined. In some embodiments, for a non-tubular model, the contour expression may be other types of equations, e.g., equations representing polygons, equations representing arcs, equations representing other regular two-dimensional closed or unclosed contours, or equations representing irregular two-dimensional closed or unclosed contours. In some embodiments, contour expressions and/or contour rotation angles corresponding to a plurality of reconstruction planes may be the same or different. For example, a contour expression corresponding to a reconstruction plane A and a contour expression corresponding to a reconstruction plane B may be both elliptic equations, but a parameter a and/or a parameter b may be different. As another example, the reconstruction plane A and the reconstruction plane B may correspond to the same elliptic equations, but different contour rotation angles. As yet another example, the contour expression corresponding to the reconstruction plane A may be the elliptic equation, and the contour expression corresponding to the reconstruction plane B may be an equation representing a rectangle.

The processor 101 may perform contour point sampling based on the contour expression, and a count of the sampled contour points may be preset. For example, the processor 101 may perform contour point sampling on a two-dimensional contour corresponding to the contour expression in the reconstruction plane. In some embodiments, processor 101 may perform uniform sampling such that a distribution of the sampled contour points may be uniform. The contour points sampled in the reconstruction plane along the contour expression may be referred to as an initial two-dimensional contour point cloud. The processor 101 may rotate the initial two-dimensional contour point cloud around a center point corresponding to the reconstruction plane by a preset angle (i.e., the contour rotation angle) within the reconstruction plane to obtain a final two-dimensional contour point cloud.

In some embodiments, the processor 101 may determine a count of sampled contour points based on the complexity of the structure of the object (e.g., the three-dimensional object, etc.). For example, if the overall structure of the object is relatively complex, the count of sampled contour points may be relatively large. If the overall structure of the object is relatively simple, the count of sampled contour points may be relatively small. As another example, if the structure of a portion of the object corresponding to a reconstruction plane is relatively complex, the count of sampled contour points in the reconstruction plane may be relatively large. If the structure of a portion of the object corresponding to a reconstruction plane is relatively simple, the count of sampled contour points in the reconstruction plane may be relatively small.

In some embodiments, the sampled contour points may be uniformly distributed (at equal distances) or non-uniformly distributed. For example, for a relatively complex portion of the structure to be simulated, the distribution of the sampled contour points of the corresponding portion may be relatively dense. For a relatively simple portion of the structure to be simulated, the distribution of the sampled contour points of the corresponding portion may be relatively sparse. As another example, if the overall structure of the object is relatively complex, the distribution of the sampled contour points may be relatively dense. If the overall structure of the object is relatively simple, the distribution of the sampled contour points may be relatively sparse.

In embodiments of the present disclosure, sampling the contour points based on structural complexity allows the contour points to be closer to the structure of the object, which results in a more accurate grid model.

In S330, the grid model generation system 100 may generate the three-dimensional contour point cloud based on the plurality of two-dimensional contour point clouds.

In some embodiments, after generating the plurality of two-dimensional contour point clouds in each of the reconstruction planes, the processor 101 may generate the three-dimensional contour point cloud by stacking the plurality of two-dimensional contour point clouds. The generated three-dimensional contour point cloud may be further configured to generate the grid model.

In the embodiments of the present disclosure, the three-dimensional contour point cloud may include the plurality of two-dimensional contour point clouds, which makes the subsequent generation of the grid model more simplified and orderly, thereby further improving the efficiency of the generating the grid model.

It should be noted that the above process of generating the three-dimensional contour point cloud is relatively applicable to a grid model of a tubular shape, or a grid model representing a virtual structure of a tubular shape. Exemplarily, in the medical field, the virtual structure of the tubular shape may be a blood vessel, a trachea, an intestine, or the like. In other fields, the virtual structure of a tubular shape may be a cup, a pipe, a lamp, or the like. For the grid model representing the virtual structure of the tubular shape, size parameters (e.g., the parameter a and the parameter b in the elliptic equation) and contour rotation angles of openings at two ends of the structure may be determined, and then, based on a fitting algorithm (e.g., linear fitting, exponential fitting, etc.), the size parameters and the contour rotation angle of an intermediate section may be fitted, where the size parameters may be used to determine the contour expression. For the grid model representing a non-tubular structure, more size parameters may be obtained to determine the contour expression and contour rotation angle of each of the plurality of two-dimensional contours separately. Although the process is relatively complex, it does not affect the realization of the embodiment.

It should be noted that the above description of the process 300 is intended to be exemplary and illustrative only, and does not limit the application scope of the present disclosure. For those skilled in the art, various corrections and changes may be made to the process 300 under the guidance of the present disclosure. However, these corrections and changes remain within the scope of the present disclosure.

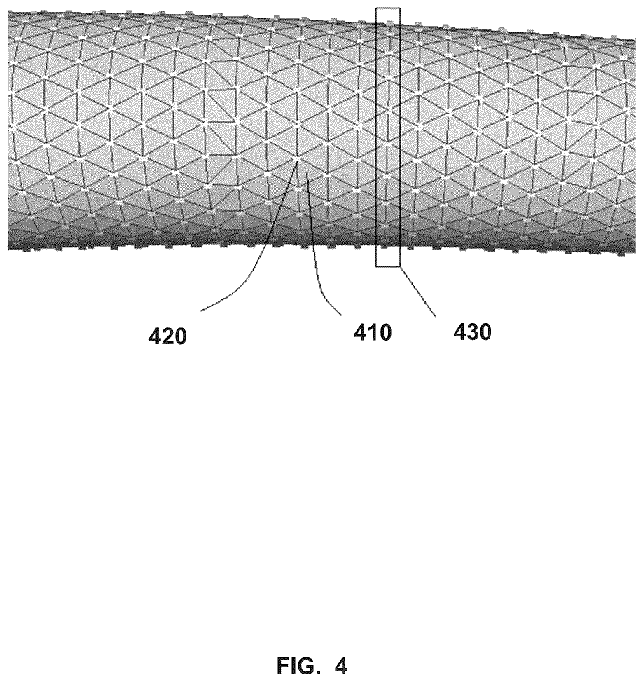

To understand a relationship between the plurality of two-dimensional contour point clouds and the three-dimensional contour point cloud in the embodiments of the present disclosure, FIG. 4 is illustrated as an example. FIG. 4 is a diagram illustrating an exemplary grid model of a tubular shape generated by a grid model generation method according to some embodiments of the present disclosure. FIG. 4 is a side view of a grid model of a tubular shape, which has an approximately horizontal straight line at the centerline (not shown in the figure). A contour point 420 may be an intersection point of lines, and a triangular section 410 may be formed by intersection of the lines. The plurality of two-dimensional contour point clouds may usually be of an annular structure (contour points may be distributed in a ring) and is usually perpendicular to the centerline of the grid model of the tubular structure, and in FIG. 4, each column of contour points 420 distributed approximately vertically may constitute half of the two-dimensional contour point cloud 430 (the other half is obscured). The three-dimensional contour point cloud of the grid model of the tubular shape in FIG. 4 may include the plurality of two-dimensional contour point clouds 430 stacked in an approximately horizontal direction.

In some embodiments, the grid model may be configured to represent a virtual blood vessel. In the medical field, the virtual blood vessel may be generated using a grid model generation method in the embodiments of the present disclosure, which has the advantage of a high generation efficiency as compared to other methods for generating the virtual blood vessel. A reconstructed three-dimensional structure and surface grid of the blood vessel may be uniquely used in radiation treatment planning, radiological interventions, multimodal image fusion, and computer-aided teaching. More importantly, the method of embodiments of the present disclosure may be used to perform a quantitative measurement on parameters related to the blood vessel and abnormity (e.g., diameter size, vessel length, cross-sectional area, stenosis rate, flow reserve fraction, etc.) and to study a correlation thereof, to derive a general rule, which may be applied in clinical diagnosis and treatment practice.

Specifically, in an application of simulating an abnormity of the blood vessel, the size parameters of part of the virtual blood vessel may be adjusted to adjust an expression of at least one two-dimensional contour point cloud, and thus adjust a distribution of the two-dimensional contour point cloud point cloud of a corresponding part. By adjusting the size parameters as described above, the virtual blood vessel may be locally narrow to simulate a corresponding abnormity. In some embodiments, the generated grid model may be displayed through, e.g., the input/output device 104. A user may adjust, through the displayed grid model, a contour expression and/or a contour rotation angle of at least one two-dimensional contour point cloud to simulate an abnormity of the blood vessel. For example, the user may adjust, through the mouse, the keyboard, or the touch screen, the location of at least one contour point of a two-dimensional contour point cloud of the displayed grid model, and/or rotate the two-dimensional contour point cloud of the displayed grid model. As another example, the user may input an adjusted contour expression and/or an adjusted contour rotation angle of a two-dimensional contour point cloud.

FIG. 5A is a flowchart illustrating an exemplary process for generating a grid section according to some embodiments of the present disclosure. In some embodiments, the process 500 may be implemented in the grid model generation system 100 illustrated in FIG. 1. For example, the process 500 may be stored in a storage medium (e.g., the storage device 102) as a form of instructions, and can be invoked and/or executed by the grid model generation system 100 (e.g., the processor 101, or one or more modules illustrated in FIG. 13). The operations of the illustrated process 500 presented below are intended to be illustrative. In some embodiments, the process 500 may be accomplished with one or more additional operations not described, and/or without one or more of the operations discussed. Additionally, the order in which the operations of the process 500 as illustrated in FIG. 5A and described below is not intended to be limiting.

In S510, the grid model generation system 100 may determine, based on two adjacent two-dimensional contour point clouds, a first two-dimensional contour point cloud and a second two-dimensional contour point cloud, each of which includes a plurality of contour points.

In some embodiments, the processor 101 may designate at least a part of contour points in one of the two adjacent two-dimensional contour point clouds as the first two-dimensional contour point cloud, and at least a part of contour points in the other two-dimensional contour point cloud as the second two-dimensional contour point cloud. It should be noted that the first two-dimensional contour point cloud and the second two-dimensional contour point cloud may be used primarily to distinguish the two adjacent two-dimensional contour point clouds, and do not represent a specific order.

In some embodiments, for a two-dimensional contour point cloud, not every contour point needs to be involved in the generation of grid sections. For example, for a three-dimensional contour point cloud obtained from an external device, the contour points may be densely distributed. To simplify the processing, for a particular two-dimensional contour point, a part of the contour points may be selected for the generation of grid sections. Merely by way of example, for a two-dimensional contour point cloud, there are contour points 1-6, and contour points 1, 3, and 5 may be selected to be the first two-dimensional contour point cloud or the second two-dimensional contour point cloud for the generation of grid sections. At this time, adjacent contour points may be contour points 1 and 3, 3 and 5, and 5 and 1.

In S520, the grid model generation system 100 may determine a plurality of first contour edges based on two adjacent contour points of the plurality of contour points in the first two-dimensional contour point cloud, and determine a first contour point in the second two-dimensional contour point cloud for each of the plurality of first contour edges.

A first contour edge refers to an edge formed by connecting two adjacent contour points in the first two-dimensional contour point cloud. In some embodiments, the processor 101 may determine the plurality of first contour edges based on any two adjacent contour points in the first two-dimensional contour point cloud. For example, a first contour edge may be obtained by connecting two adjacent contour points in the first two-dimensional contour point cloud. Because there are a plurality of contour points in each two-dimensional contour point cloud, the processor 101 may determine a plurality of first contour edges. For each first contour edge in the plurality of first contour edges, the processor 101 may determine a first contour point in the second two-dimensional contour point cloud that corresponds to the first contour edge.

In S530, the grid model generation system 100 may generate a plurality of first triangular sections based on the plurality of first contour edges and the first contour point corresponding to each of the plurality of first contour edges.

A first triangular section refers to a grid section that corresponds to a first contour edge and a first contour point. In some embodiments, the processor 101 may generate a first triangular section by connecting the first contour point in the second two-dimensional contour point cloud to two ends of the first contour edge in the first two-dimensional contour point cloud, respectively. In some embodiments, the processor 101 typically needs to iterate through all of the contour points in the first two-dimensional contour point cloud and connect any two adjacent contour points to obtain the plurality of first contour edges to enable all of the contour points in the first two-dimensional contour point cloud to be involved in the generation of grid sections. Further, connecting only two adjacent contour points to form the first contour edges avoids partial overlapping of all contour edges in one two-dimensional contour point cloud.

In 540, the grid model generation system 100 may determine a plurality of reconstructed contour points in the plurality of contour points of the second two-dimensional contour point cloud.

In some embodiments, before determining a second contour edge, the processor 101 may determine the plurality of reconstructed contour points in the second two-dimensional contour point cloud. A reconstructed contour point refers to a contour point that is designated as the first contour point in the second two-dimensional contour point cloud. In some embodiments, in the second two-dimensional contour point cloud, only contour points that have been designated as the first contour points can participate in the reconstruction of the second triangular section.

In S550, the grid model generation system 100 may determine at least one second contour edge based on two adjacent reconstructed contour points in the plurality of reconstructed contour points and determine a second contour point in the plurality of contour points of the first two-dimensional contour point cloud corresponding to each second contour edge.

The second contour edge refers to an edge formed by connecting two adjacent reconstructed contour points in the second two-dimensional contour point cloud. The second contour edge and the second contour point may be determined similarly as the first contour point and the first contour edge. For example, the processor 101 may determine the second contour edge by connecting any two adjacent reconstructed contour points in the second two-dimensional contour point cloud. For each second contour edge, the processor 101 may determine a second contour point corresponding to the second contour edge in the first two-dimensional contour point cloud.

In S560, the grid model generation system 100 may generate at least one second triangular section based on the at least one second contour edge and the second contour point corresponding to each second contour edge.

The second triangular section refers to a grid section corresponding to the second contour point and the second contour edge. After the plurality of first triangular sections are generated, the at least one second triangular section may be generated similarly. For example, the processor 101 may generate the second triangular section by connecting the second contour edge in the second two-dimensional contour point cloud to each of the ends of the second contour point in the first two-dimensional contour point cloud. The processor 101 typically needs to iterate through all of the reconstructed contour points in the second two-dimensional contour point cloud, connect any two adjacent reconstructed contour points to form a plurality of second contour edges, to enable all of the reconstructed contour points to be involved in the reconstruction of the grid model.

It should be noted that when determining the first contour point corresponding to the first contour edge, and determining the second contour point corresponding to the second contour edge, it is necessary to ensure that triangular sections with partially overlapping areas are not ultimately generated.

In some embodiments, the first contour point refers to a contour point in the second two-dimensional contour point cloud closest to the corresponding first contour edge, and the second contour point refers to a common vertex of two first triangular sections that respectively include the two reconstructed contour points of the second contour edge (e.g., the two adjacent reconstructed contour points based on which the second contour edge is determined, that is, the two adjacent reconstructed contour points that are connected to determine the second contour edge). Specifically, the embodiments of the present disclosure also provide a process for determining the first contour point and the second contour point, as described below.

In some embodiments, the first triangular section may include the first contour edge and the first contour point. After the first contour edge is determined in the first two-dimensional contour point cloud, the contour point closest to the first contour edge in the second two-dimensional contour point cloud may be designated as the first contour point. It should be noted that a distance between a contour point and a contour edge is a straight-line distance between the contour point and a specific point in the contour edge. The specific point may be a midpoint, a left endpoint, a right endpoint, a quarter point, etc., of the contour edge, or a perpendicular distance between the contour point and the contour edge, as long as ensuring the same definition of the specific point in any contour edge, e.g., the specific point of any contour edge is the midpoint. Determining the first contour point using the above manner may ensure that any two first triangular sections may not be partially overlapped, or that no edges may cross (except for edge endpoints). Exemplarily, after a first triangular section T1 is generated, in a process of further generating a new first triangular section T2 to the left of the first triangular section T1, a new first contour edge E2 corresponding to the new first triangular section T2 may be determined adjacent to the left of the first contour edge E1 corresponding to the first triangular section T1, and accordingly, a new first contour point P2 corresponding to the new first triangular section T2 may be determined. Because the new first contour edge E2 is to the left of the first contour edge E1, according to the principle of selecting the first contour point with the closest distance, the new first contour point P2 cannot be to the right of the first contour point P1 corresponding to the first triangular section T1, and the new first contour point P2 is either the first contour point P1 or a contour point to the left of the first contour point P1. Thus, the newly generated first triangular section T2 does not partially overlap with the first triangular section T1.

It should be noted that when there are at least two contour points in the second two-dimensional contour point cloud that are closest to the first contour edge, one of the at least two contour points that does not cause partial overlapping of the first triangular sections may be selected as the first contour point.

In some embodiments, the second triangular section may include the second contour edge and the second contour point. After the second contour edge is determined in the second two-dimensional contour point cloud, the determination of the second contour point in the first two-dimensional contour point cloud may be relatively important, and it is necessary to ensure that any two second triangular sections, and the second triangular sections and the first triangular sections do not partially overlap. First, the processor 101 may determine at least one first triangular section (denoted as a first set of triangular sections) that uses one endpoint of the second contour edge as a vertex, and determine at least one first triangular section (denoted as a second set of triangular sections) that uses the other endpoint of the second contour edge as a vertex. Then, the processor 101 may determine whether there is a first triangular section in the first set of triangular sections that has a common vertex with a first triangular section in the second set of triangular sections. If so, the common vertex may be designated as the second contour point. The second triangular section generated in this manner does not partially overlap with the first triangular section. For the two first triangular sections having the common vertex, an edge of one first triangular section connecting the common vertex and an endpoint of the second contour edge and an edge of the other first triangular section connecting the common vertex and the other endpoint of the second contour edge may form two edges of the second triangular section.

It should be noted that all the contour points in the second two-dimensional point cloud may not be ensured to participate in the generation of the second triangular section. This is because not all the contour points in the second two-dimensional point cloud can participate in the generation of the first triangular section. As a result, contour points in the second two-dimensional contour point cloud that do not participate in the generation of the first triangular section may also be unable to participate in the generation of the second triangular section. Particularly, when a count of contour points in the second two-dimensional contour point cloud is greater than a count of contour points in the first two-dimensional contour point cloud, the proportion of the contour points in the second two-dimensional contour point cloud that do not participate in the generation of the triangular section may be relatively high.

To improve stability of the grid model, in some embodiments, the count of contour points in the first two-dimensional contour point cloud may be greater than the count of contour points in the second two-dimensional contour point cloud. That is, after determining the two adjacent contour point clouds, the first contour edge may be first determined in the 2D contour point cloud with a larger count of contour points and the first contour point may be determined in the 2D contour point cloud with a smaller count of contour points, thereby generating the first triangular section. Then, the second contour edge may be determined in the contour point cloud with a smaller count of contour points and the second contour point may be determined in the contour point cloud with a larger count of contour points, thereby generating the second triangular section.

In some embodiments, the contour points in the second two-dimensional contour point cloud that are not involved in the generation of the first triangular section may also be involved in the generation of the second triangular section. In some embodiments, the processor 101 may generate the second triangular section based on candidate contour points. FIG. 5B is a schematic diagram illustrating generating a grid section by candidate contour points according to some embodiments of the present disclosure. For example, as shown in FIG. 5B, the processor 101 may determine a candidate contour point a in a second two-dimensional contour point cloud. The candidate contour point a may be a contour point in the second two-dimensional contour point cloud that is not involved in the generation of the first triangular section. The processor 101 may determine a third contour point d in the first two-dimensional contour point cloud based on a contour point b and a contour point c in the second two-dimensional contour point cloud adjacent to the candidate contour point a. The third contour point refers to a common vertex of two first triangular sections including the contour points b and c respectively. The processor 101 may generate the second triangular section based on the third contour point d and the candidate contour point a. For example, the third contour point d and the candidate contour point a may be connected to generate a second triangular section abd and a second triangular section acd. In some embodiments, the processor 101 may determine at least one candidate contour point and a third contour point corresponding to each of the at least one candidate contour point, thereby generating a plurality of second triangular sections based on the at least one candidate contour point and the at least one third contour point.

In the embodiments of the present disclosure, the contour points that are not involved in the generation of the first triangular section may also participate in the generation of the second triangular section, which allows more contour points to be involved in reconstruction of the grid model and improves accuracy of the grid model reconstruction.

In some embodiments, when generating the grid model representing a virtual structure of a tubular shape, a blocking operation may be performed on the grid sections. That is, blocking grid sections may be generated in the head-end two-dimensional contour point cloud and the tail-end two-dimensional contour point cloud in the grid model of the tubular shape.

For example, taking the head-end two-dimensional contour point cloud as an example, the processor 101 may connect any adjacent contour points in the head-end two-dimensional contour point cloud. The processor 101 may determine a third point. The processor 101 may connect the contour points in the two-dimensional contour point cloud to the third point respectively to generate the blocking grid sections. The third point may be located in the corresponding reconstruction plane and located on a contour corresponding to the corresponding contour expression or within a region enclosed by the contour corresponding to the corresponding contour expression. Preferably, the third point may be a center point corresponding to the head-end two-dimensional contour point cloud. The generation of the blocking gird sections on the tail-end two-dimensional contour point cloud may be similar to the generation of the blocking gird sections on the head-end two-dimensional contour point cloud.

In some embodiments, after generating the triangular sections between two adjacent two-dimensional contour point clouds, the processor 101 may combine a first triangular section and a second triangular section adjacent to each other to form a quadrangular section. It is necessary to ensure that the finally generated grid sections (e.g., the triangular sections and/or the quadrangular sections) do not overlap with each other. In FIG. 5B, for example, first triangular sections kji, jhi, hfg, fdc, and deb, and second triangular sections igh, gcf, cad, and abd, are generated between two adjacent two-dimensional contour point clouds. The first triangular section jhi and the second triangular section igh is combined to form a quadrangular section jhgi, the first triangular section hfg and the second triangular section gcf is combined to form a quadrangular section hfcg, the first triangular section fdc and the second triangular section cad is combined to form a quadrangular section fdac, and the first triangular section deb and the second triangular section abd is combined to form a quadrangular section deba. Finally, the quadrangular sections jhgi, hfcg, fdac, deba, and the triangular section kji are generated between the two adjacent two-dimensional contour point clouds.

The above process for generating the grid sections and generating the grid model based on the grid sections has an advantage of high efficiency.

It should be noted that the above description of the process 500 is intended to be exemplary and illustrative only and does not limit the application scope of the present disclosure. For those skilled in the art, various corrections and changes may be made to the process 500 under the guidance of the present disclosure. However, these corrections and changes are still within the scope of the present disclosure.

FIG. 6 is a flowchart illustrating an exemplary process for determining two-dimensional contour point clouds according to some embodiments of the present disclosure. In some embodiments, the process 600 may be implemented in the grid model generation system 100 illustrated in FIG. 1. For example, the process 600 may be stored in a storage medium (e.g., the storage device 102) as a form of instructions, and can be invoked and/or executed by the grid model generation system 100 (e.g., the processor 101, or one or more modules illustrated in FIG. 13). The operations of the illustrated process 600 presented below are intended to be illustrative. In some embodiments, the process 600 may be accomplished with one or more additional operations not described, and/or without one or more of the operations discussed. Additionally, the order in which the operations of the process 600 as illustrated in FIG. 6 and described below is not intended to be limiting.

In some embodiments, the process 600 may be executed by the processor 101, and the process 600 may be used to implement operation S220.

As shown in FIG. 6, the process 600 includes the following operations.

In S610, the grid model generation system 100 may obtain a centerline of an object based on a three-dimensional contour point cloud. The centerline may include a plurality of center points.

Descriptions regarding the three-dimensional contour point cloud and the centerline may be found in FIGS. 2 and 3 and related descriptions thereof, which may not be repeated here. In some embodiments, the processor 101 may determine a count of center points based on an overall density of the three-dimensional contour point cloud. For example, if the overall density of the three-dimensional contour point cloud is large, the count of center points in the centerline may be relatively large. If the overall density of the three-dimensional contour point cloud is small, the count of center points may be relatively small.

In S620, the grid model generation system 100 may determine a reconstruction plane at each of the plurality of center points.

In some embodiments, for each center point in the plurality of center points, the processor 101 may determine a tangent vector of the centerline at the center point, thereby determining a reconstruction plane that is perpendicular to the tangent vector and passes through the center point. For the plurality of center points in the centerline, the processor 101 may determine a plurality of reconstruction planes. More descriptions regarding determining the plurality of reconstruction planes may be found in FIG. 3 and related description thereof.

In S630, for each of the plurality of two-dimensional contour point clouds, the grid model generation system 100 may determine the two-dimensional contour point cloud based on one or more contour points in the three-dimensional contour point cloud that have a distance from the corresponding reconstruction plane less than a threshold. Descriptions regarding the plurality of two-dimensional contour point clouds may be found in FIGS. 2 and 3 and related descriptions thereof.

In some embodiments, a two-dimensional contour point cloud may or may not be located in the same plane. The processor 101 may determine one or more contour points in the three-dimensional contour point cloud that have a distance from the reconstruction plane less than the threshold as being located on one layer structure, and determine a two-dimensional contour point cloud based on the one or more contour points that are located on the layer structure.

For example, if the threshold is set to 0, the processor 101 may determine one or more contour points in the three-dimensional contour point cloud that are located in the reconstruction plane as being located on a layer structure. In this case, the layer structure may be a plane. As another example, if the threshold is set to be greater than 0, in this case, the layer structure is a non-plane (e.g., a curved surface, a folded surface, etc.). The threshold may be set empirically or adjusted based on real conditions. The threshold corresponding to each of the plurality of reconstruction planes may be the same or different. For example, for a reconstruction plane, a corresponding threshold may be determined based on a distance between the reconstruction plane and an adjacent reconstruction plane (e.g., the threshold is positively correlated with the distance). The corresponding threshold may also be determined based on a smaller distance in distances between the reconstruction plane and two adjacent reconstruction planes (e.g., the threshold is positively correlated with the smaller distance). The corresponding threshold may also be determined based on an average of the distances between the reconstruction plane and the two adjacent reconstruction planes (e.g., the threshold is positively correlated with the average of the distances).

In some embodiments, a count of contour points located on the layer structure needs to be determined. If the count of contour points in the layer structure is two or more, the processor 101 may determine the plurality of contour points in the corresponding layer structure to be a two-dimensional contour point cloud. If there is only one contour point in the layer structure, the contour point may be excluded and the contour point may not be involved in the reconstruction of grid sections, i.e., the layer structure with only one contour point may not be determined as a two-dimensional contour point cloud.

In embodiments of the present disclosure, since grid sections are generated between two adjacent two-dimensional contour point clouds, an ordering degree of the three-dimensional contour point cloud may be required. For example, in order to determining a plurality of stacked two-dimensional contour point clouds, it requires that the majority of the contour points in the three-dimensional contour point cloud falls into a plurality of layer structures. For the three-dimensional contour point cloud obtained from an external device, the three-dimensional contour point cloud obtained by a sensor, the three-dimensional contour point cloud obtained by a three-dimensional model, and the three-dimensional contour point cloud obtained by medical imaging data, the ordering degree thereof cannot be guaranteed. If it requires that the three-dimensional contour point cloud is divided into a plurality of two-dimensional contour point clouds in each of which the contour points are located in the same plane, there may be a certain count of contour points that cannot be classified into the plurality of two-dimensional contour point clouds and thus cannot participate in the reconstruction of the grid model. If it is not required that the contour points in the two-dimensional contour point cloud are located in the same plane, there may be fewer requirements for the ordering degree of the three-dimensional contour point cloud. Thus, the method for generating the grid model in the embodiments of the present disclosure can be applied to more three-dimensional contour point clouds, and more contour points may be involved in the reconstruction of the grid model, thereby improving compatibility and accuracy of the grid model reconstruction method.

It should be noted that the above description of the process 600 is intended to be exemplary and illustrative only and does not limit the application scope of the present disclosure. For those skilled in the art, various corrections and changes may be made to the process 600 under the guidance of the present disclosure. However, these corrections and changes remain within the scope of the present disclosure.

The following is an example of a grid model generation method for generating a virtual blood vessel in an embodiment of the present disclosure to further illustrate the method.

FIG. 7 is a flowchart illustrating an exemplary process for generating a grid model according to some other embodiments of the present disclosure.

In some embodiments, the process 700 may be implemented in the grid model generation system 100 illustrated in FIG. 1. For example, the process 700 may be stored in a storage medium (e.g., the storage device 102) as a form of instructions, and can be invoked and/or executed by the grid model generation system 100 (e.g., the processor 101, or one or more modules illustrated in FIG. 13). The operations of the illustrated process 700 presented below are intended to be illustrative. In some embodiments, the process 700 may be accomplished with one or more additional operations not described, and/or without one or more of the operations discussed. Additionally, the order in which the operations of the process 700 as illustrated in FIG. 7 and described below is not intended to be limiting.

In S710, the grid model generation system 100 may obtain a centerline of a blood vessel to be simulated and a tangent vector for each of a plurality of center points on the centerline.

More descriptions regarding the centerline may be found in FIG. 3 and related descriptions thereof. In some embodiments, if an object (e.g., a three-dimensional object) is a virtual blood vessel, the processor 101 may obtain at least one center point input by a user, and an interpolation process may be performed on the at least one center point to obtain a set of dense center points. The centerline may be obtained by connecting the set of dense center points. The interpolation process may include algorithms such as B-spline interpolation, bilinear interpolation, etc. In some embodiments, the processor 101 may obtain an analytical expression for each center point in the plurality of center points based on an interpolation algorithm, and determine a tangent vector for each center point. The tangent vector of the center point refers to a tangent vector of the centerline at the center point.

In S720, the grid model generation system 100 may determine a local coordinate system for each center point in the plurality of center points.

The local coordinate system for the center point refers to a local coordinate system centered on the center point (taking the center point as an origin of the local coordinate system). In some embodiments, for each center point, the processor 101 may determine a reconstruction plane at the center point for reconstructing a contour of the virtual blood vessel. The reconstruction plane perpendicular to the tangent vector of the center point may be set as a uv-plane, the tangent vector of the center point may be set as a w-axis, and any vector in the uv-plane may be set as a u-axis. The w-axis and the u-axis may make a cross-product operation to obtain a v-axis, thereby determining the local coordinate system of the center point.

In S730, the grid model generation system 100 may obtain a contour at each center point.

In some embodiments, the processor 101 may obtain a contour at each center point based on a randomized algorithm. For example, the processor 101 may obtain a long axis size, a short axis size, and a contour rotation angle corresponding to the first center point, and a long axis size, a short axis size, and a contour rotation angle corresponding to the last center point based on the randomized algorithm. The processor 101 may obtain the long axis size, the short axis size, and the contour rotation angle at each center point based on the long axis sizes, the short axis sizes, and the contour rotation angles corresponding to the first and the last center points, and a fitting algorithm such as linear fitting, exponential fitting, or the like. The processor 101 may determine a contour expression of each center point based on the long axis size a and the short axis size b at each center point and rotate the contour expression accordingly in the uv plane based on the contour rotation angle. In some embodiments, the contour expression may be expressed as: x²/a²+y²/b²=1. Where x and y denote coordinates of the contour points in the uv plane.

In S740, the grid model generation system 100 may obtain a plurality of contour points to generate the 2D contour point clouds by sampling a contour corresponding to each center point.

In some embodiments, the processor 101 may obtain a count of sampled contour points corresponding to each center point and uniformly sample the contour points based on the contour expression and the rotation angle to obtain the 2D contour point clouds.

In S750, the grid model generation system 100 may generate triangular sections between two adjacent 2D contour point clouds, thereby finishing the reconstruction of a three-dimensional surface of the virtual blood vessel.

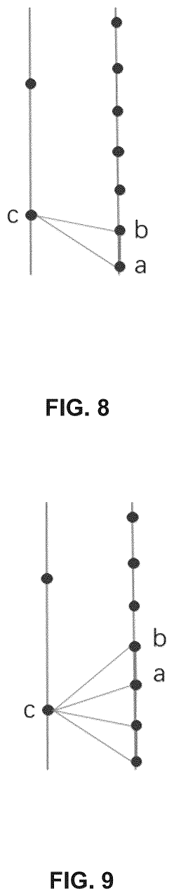

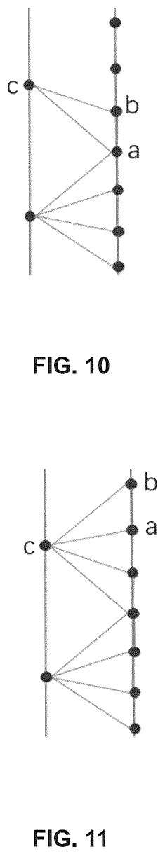

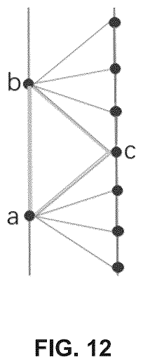

The process for generating grid sections may be described in conjunction with FIGS. 8-12, and the process for generating grid sections may include the following operations.
a. A relatively sparse two-dimensional contour point cloud (e.g., a first two-dimensional contour point cloud) and a relatively dense 2D contour point cloud (e.g., a second two-dimensional contour point cloud) may be determined. The two columns of contour points in FIGS. 8-12 are two adjacent 2D contour point clouds, wherein the 2D contour point cloud with less contour points on the left side is the first two-dimensional contour point cloud, and the 2D contour point cloud with more contour points on the right side is the second two-dimensional contour point cloud.
b. Starting from the first two-dimensional contour point cloud, two adjacent contour points may be selected and noted as a and b, a contour point closest to the line segment ab may be selected in the second two-dimensional contour point cloud and noted as point c, a directed first triangular section abc may be determined, and the operation may be stopped until any two adjacent contour points in the first two-dimensional contour point cloud have been selected. Exemplarily, referring to FIG. 8, from bottom to top, the first and second points of the first two-dimensional contour point cloud are selected as points a and b, and the first point of the second two-dimensional contour point cloud is selected as point c. Referring to FIG. 9, from bottom to top, the third and fourth points of the first two-dimensional contour point cloud are selected as points a, and b, and the first point of the second two-dimensional contour point cloud is selected as point c. Referring to FIG. 10, from bottom to top, the fourth and fifth points of the first two-dimensional contour point cloud are selected as points a, and b, and the second point of the second two-dimensional contour point cloud is selected as point c. Referring to FIG. 11, from bottom to top, the sixth and seventh points of the first two-dimensional contour point cloud are selected as points a, and b, and the second point of the second two-dimensional contour point cloud is selected as point c.
c. It is the turn of the second two-dimensional contour point cloud. Two contour points in the second two-dimensional contour point cloud may be selected and noted as a and b. From a determined first triangular section, a first edge including point a is determined. From another determined first triangular section, a second edge including point b is determined. The first edge and the second edge have a common vertex c. A directed second triangular section cba may be determined. Referring to FIG. 12, when points a and b are determined in the second two-dimensional contour point cloud, there are four line segments including point a and there are four line segments including point b. One of the four line segments including point a may be chosen and one of the four line segments including point b may be chosen, wherein the selected two line segments intersect at a point, and the point may be designated as point c.
d. Any two adjacent 2D contour point clouds may go through the above process to reconstruct a surface of the virtual blood vessel.
e. Blocking grid sections may be generated in the head-end two-dimensional contour point cloud and the tail-end two-dimensional contour point cloud. For example, taking the head-end two-dimensional contour point cloud as an example, the processor 101 may connect any adjacent contour points in the head-end two-dimensional contour point cloud. The processor 101 may determine a third point. The processor 101 may connect the contour points in the two-dimensional contour point cloud to the third point respectively to generate the blocking grid sections. The generation of the blocking gird sections on the tail-end two-dimensional contour point cloud may be similar to the generation of the blocking gird sections on the head-end two-dimensional contour point cloud.

In embodiments of the present disclosure, reconstructing the grid model (e.g., a virtual blood vessel contour) by the above process may require less ordering degree of the three-dimensional contour point cloud and have greater compatibility for the three-dimensional contour point cloud.

In S760, the grid model generation system 100 may create narrow areas on the three-dimensional surface to simulate an abnormity.

In some embodiments, the processor 101 may first determine at least one 2D contour point cloud that need to be added to an abnormality narrowing, and then adjust a contour expression of the at least one 2D contour point cloud to make the at least one 2D contour point cloud narrow, which causes the virtual blood vessel to become locally narrow, thereby simulating the abnormity of the virtual blood vessel.

The embodiments of the present disclosure also provide a grid model generation system for realizing any of the embodiments and implementations of the present disclosure, which may not be repeated where descriptions have already been made. As used hereinafter, the terms “module”, “unit”, “subunit”, etc. are combinations of software and/or hardware that perform a preset function. While the device described in the following embodiments is preferably implemented in software, the implementation of hardware, or a combination of software and hardware, is also possible and contemplated.

FIG. 13 is a diagram illustrating exemplary modules of a grid model generation system according to some embodiments of the present disclosure. In some embodiments, the grid model generation system 1300 may be implemented based on the processor 101. As shown in FIG. 13, the grid model generation system 1300 may include a point cloud acquisition module 1310, a point cloud determination module 1320, a grid section generation module 1330, and a structure generation module 1340.

In some embodiments, the point cloud acquisition module 1310 may be configured to obtain a three-dimensional contour point cloud of a grid model to be generated.

In some embodiments, the point cloud acquisition module 1310 may be further configured to obtain a centerline of the grid model to be generated, and the centerline may include a plurality of center points. The point cloud acquisition module 1310 may be further configured to determine a reconstruction plane at each of the plurality of center points, generate a plurality of two-dimensional contour point clouds, and generate a three-dimensional contour point cloud based on the plurality of two-dimensional contour point clouds.

In some embodiments, the point cloud acquisition module 1310 may be further configured to obtain a contour expression and a contour rotation angle for each of the plurality of two-dimensional contour point clouds and generate the two-dimensional contour point cloud by sampling a preset count of contour points based on the contour expression and the contour rotation angle.

In some embodiments, the point cloud determination module 1320 may be configured to determine a plurality of two-dimensional contour point clouds based on the three-dimensional contour point cloud.

In some embodiments, the point cloud determination module 1320 may be further configured to obtain the centerline of the grid model to be generated based on the three-dimensional contour point cloud, and the centerline may include a plurality of center points. The point cloud determination module 1320 may be further configured to determine the reconstruction plane at each of the plurality of center points and determine the plurality of two-dimensional contour point clouds based on one or more contour points in the three-dimensional contour point cloud that have a distance from the reconstruction plane less than a threshold.

In some embodiments, the grid section generation module 1330 may be configured to generate grid sections between two adjacent two-dimensional contour point clouds based on the contour points in the plurality of two-dimensional contour point clouds.

In some embodiments, the grid section may include a triangular section or a quadrangular section.

In some embodiments, the grid section may include the triangular section, and the grid section generation module 1330 may be further configured to determine a first two-dimensional contour point cloud and a second two-dimensional contour point cloud based on two adjacent two-dimensional contour point clouds; determine a plurality of first contour edges based on two adjacent contour points in the first two-dimensional contour point cloud; determine a plurality of first contour points corresponding to the plurality of first contour edges in the second two-dimensional contour point cloud; generate a plurality of first triangular sections based on the first contour points and the first contour edges; determine a plurality of reconstructed contour points in the second two-dimensional contour point cloud; determine a second contour edge based on two adjacent reconstructed contour points; determine a second contour edge corresponding to the second contour point in the first two-dimensional contour point cloud; and generate a second triangular section based on the second contour point and the second contour edge.

In some embodiments, the first contour points refer to a contour point in the second two-dimensional contour point cloud that is closest to the first contour edge. The second contour point refers to a common vertex of two first triangular sections including two endpoints of the second contour edge, respectively. In some embodiments, a count of contour points of the first contour point cloud may be greater than a count of contour points of the second contour point cloud.

In some embodiments, the structure generation module 1340 may be configured to generate the grid model based on the grid sections. In some embodiments, the grid model may be used to represent a virtual blood vessel, and the structure generation module 1340 may be further configured to select at least one two-dimensional contour point cloud, adjust the contour expression of each of the at least one two-dimensional contour point cloud to simulate an abnormity of the virtual blood vessel.

It should be understood that the device shown in FIG. 13 and its modules may be realized using various manners.

It should be noted that the above description of the grid model generation system and its modules is provided only for descriptive convenience, and does not limit the present disclosure to the scope of the cited embodiments. It may be understood that those skilled in the art, after understanding the principle of the system, may arbitrarily combine the modules or form a sub-system to be connected to the other modules without departing from the principle. In some embodiments, the point cloud acquisition module, the point cloud determination module, the grid section generation module, and the structure generation module disclosed in FIG. 13 may be different modules in one device, or one module to realize the functions of two or more of the above-mentioned modules. For example, each of the modules may share a common storage module, and each of the modules may have its storage module. Such modifications are within the protection scope of the present disclosure.

With the above process for generating the grid model, the three-dimensional contour point cloud of the grid model may be first obtained, and then the plurality of two-dimensional contour point clouds may be determined based on the three-dimensional contour point cloud, and the grid sections may be generated between any two adjacent two-dimensional contour point clouds. Generating the grid sections between two adjacent two-dimensional contour point clouds may make the process for generating the grid model simple and neat. At the same time, the grid sections between any two adjacent two-dimensional contour point clouds may be simultaneously generated, which greatly improves efficiency of generating the grid model. In general, determining the plurality of two-dimensional contour point clouds in the three-dimensional contour point cloud and generating the grid sections between two adjacent two-dimensional contour point clouds may improve the efficiency of generating the grid model, thereby improving the efficiency of generating a virtual structure using the grid model and solving the problem that a grid model generation algorithm in the related technology is relatively complicated and the efficiency of generating the grid model is relatively low.

It should be noted that each of the above modules may be either a function module or a program module, and may be implemented either by software or by hardware. For the modules that are realized through hardware, each of the modules may be located in the same processor, or each of the modules may also be located in different processors according to any combination of forms.

An electronic device is also provided in the embodiments of the present disclosure, and the electronic device may include a storage device including computer programs and a processor configured to execute the computer programs to perform the operations in any of the above embodiments.

In some embodiments, the electronic device may further include a transmission device and an input/output device. The transmission device and the input/output device may be connected to the processor, respectively.

It should be noted that specific examples in the embodiment may be referred to the examples described in the above embodiments and optional embodiments, and may not be repeated in the embodiment.

It should be noted that user information (including, but not limited to, user device information, user personal information, etc.) and data (including, but not limited to, data used for analysis, data stored, data displayed, etc.) covered in the present disclosure are authorized by the user or fully authorized by all parties.

One or more embodiments of the present disclosure further provide a non-transitory computer-readable medium including at least one set of instructions. When executed by one or more processors of a computing device, the at least one set of instructions may cause a computing device to perform a grid model generation method in any of the above embodiments.

Having thus described the basic concepts, it may be rather apparent to those skilled in the art after reading this detailed disclosure that the foregoing detailed disclosure is intended to be presented by way of example only and is not limiting. Although not explicitly stated here, those skilled in the art may make various modifications, improvements, and amendments to the present disclosure. These alterations, improvements, and modifications are intended to be suggested by this disclosure and are within the spirit and scope of the exemplary embodiments of the present disclosure.

Moreover, certain terminology has been used to describe embodiments of the present disclosure. For example, the terms “one embodiment,”“an embodiment,” and/or “some embodiments” mean that a particular feature, structure, or feature described in connection with the embodiment is included in at least one embodiment of the present disclosure. Therefore, it is emphasized and should be appreciated that two or more references to “an embodiment” or “one embodiment” or “an alternative embodiment” in various portions of the present disclosure are not necessarily all referring to the same embodiment. In addition, some features, structures, or characteristics of one or more embodiments in the present disclosure may be properly combined.

Furthermore, the recited order of processing elements or sequences, or the use of numbers, letters, or other designations therefore, is not intended to limit the claimed processes and methods to any order except as may be specified in the claims. Although the above disclosure discusses some embodiments of the invention currently considered useful by various examples, it should be understood that such details are for illustrative purposes only, and the additional claims are not limited to the disclosed embodiments. Instead, the claims are intended to cover all combinations of corrections and equivalents consistent with the substance and scope of the embodiments of the invention. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that object of the present disclosure requires more features than the features mentioned in the claims. Rather, claimed subject matter may lie in less than all features of a single foregoing disclosed embodiment.

In some embodiments, the numbers expressing quantities or properties used to describe and claim certain embodiments of the present disclosure are to be understood as being modified in some instances by the term “about,”“approximate,” or “substantially.” For example, “about,”“approximate” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated by this reference in its entirety for all purposes. History application documents that are inconsistent or conflictive with the contents of the present disclosure are excluded, as well as documents (currently or subsequently appended to the present specification) limiting the broadest scope of the claims of the present disclosure. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. A grid model generation method implemented on a machine including one or more processing devices and one or more storage devices, comprising:
obtaining a three-dimensional (3D) contour point cloud related to an object;
determining a plurality of two-dimensional (2D) contour point clouds based on the three-dimensional contour point cloud;
generating a plurality of grid sections between two adjacent two-dimensional contour point clouds of the plurality of two-dimensional contour point clouds; and
generating a grid model of the object based on the plurality of grid sections.

2. The grid model generation method of claim 1, wherein the plurality of grid sections include a triangular section or a quadrangular section.

3. The grid model generation method of claim 1, wherein the plurality of grid sections include a triangular section; and
the generating a plurality of grid sections between two adjacent two-dimensional contour point clouds of the plurality of two-dimensional contour point clouds includes:
determining, based on the two adjacent two-dimensional contour point clouds, a first two-dimensional contour point cloud and a second two-dimensional contour point cloud each of which includes a plurality of contour points;
determining a plurality of first contour edges based on two adjacent contour points of the plurality of contour points in the first two-dimensional contour point cloud;
for each of the plurality of first contour edges, determining a first contour point in the plurality of contour points of the second two-dimensional contour point cloud;
generating a plurality of first triangular sections based on the first contour points and the first contour edges;
determining a plurality of reconstructed contour points in the plurality of contour points of the second two-dimensional contour point cloud;
determining a second contour edge based on two adjacent reconstructed contour points in the plurality of reconstructed contour points;
determining a second contour point in the plurality of contour points of the first two-dimensional contour point cloud corresponding to the second contour edge; and
generating a second triangular section based on the second contour point and the second contour edge.

4. The grid model generation method of claim 3, wherein
the first contour point is a contour point in the second two-dimensional contour point cloud closest to the corresponding first contour edge; and
the second contour point is a common vertex to two first triangular sections respectively including the two adjacent reconstructed contour points based on which the second contour edge is determined.

5. The grid model generation method of claim 3, wherein a count of the plurality of contour points in the first two-dimensional contour point cloud is greater than a count of the plurality of contour points in the second two-dimensional contour point cloud.

6. The grid model generation method of claim 1, wherein the obtaining a three-dimensional (3D) contour point cloud related to an object includes:
obtaining a centerline of the object, wherein the centerline includes a plurality of center points;
determining a reconstruction plane at each of the plurality of center points;
generating the plurality of two-dimensional contour point clouds based on the reconstruction planes; and
generating the three-dimensional contour point cloud based on the plurality of two-dimensional contour point clouds.

7. The grid model generation method of claim 6, wherein the generating the plurality of two-dimensional contour point clouds based on the reconstruction planes includes:
for each of the plurality of two-dimensional contour point clouds,
obtaining a contour expression and a contour rotation angle; and
generating the two-dimensional contour point cloud by sampling a preset count of contour points in the corresponding reconstruction plane based on the contour expression and the contour rotation angle.

8. The grid model generation method of claim 1, wherein the determining a plurality of two-dimensional (2D) contour point clouds based on the three-dimensional contour point cloud includes:
obtaining a centerline of the object based on the three-dimensional contour point cloud, wherein the centerline includes a plurality of center points;
determining a reconstruction plane at each of the plurality of center points; and
for each of the plurality of two-dimensional contour point clouds, determining the two-dimensional contour point cloud based on one or more contour points in the three-dimensional contour point cloud that have a distance from the reconstruction plane less than a threshold.

9. The grid model generation method of claim 1, wherein the grid model is configured to represent a virtual blood vessel.

10. The grid model generation method of claim 9, further comprising:
selecting at least one of the plurality of two-dimensional contour point clouds; and
adjusting a contour expression of the at least one of the plurality of two-dimensional contour point clouds to simulate an abnormity of the virtual blood vessel.

11. A grid model generation system, comprising:
at least one storage device including a set of instructions; and
at least one processing device in communication with the at least one storage device, wherein when executing the set of instructions, the at least one processing device is directed to perform operations including:
obtaining a three-dimensional (3D) contour point cloud related to an object;
determining a plurality of two-dimensional (2D) contour point clouds based on the three-dimensional contour point cloud;
generating a plurality of grid sections between two adjacent two-dimensional contour point clouds of the plurality of two-dimensional contour point clouds; and
generating a grid model of the object based on the plurality of grid sections.

12. The grid model generation system of claim 11, wherein the plurality of grid sections include a triangular section or a quadrangular section.

13. The grid model generation system of claim 11, wherein the grid surface is a triangular surface; and
the generating a plurality of grid sections between two adjacent two-dimensional contour point clouds of the plurality of two-dimensional contour point clouds includes:
determining, based on the two adjacent two-dimensional contour point clouds, a first two-dimensional contour point cloud and a second two-dimensional contour point cloud each of which includes a plurality of contour points;
determining a plurality of first contour edges based on two adjacent contour points of the plurality of contour points in the first two-dimensional contour point cloud;
for each of the plurality of first contour edges, determining a first contour point in the plurality of contour points of the second two-dimensional contour point cloud;
generating a plurality of first triangular sections based on the first contour points and the first contour edges;
determining a plurality of reconstructed contour points in the plurality of contour points of the second two-dimensional contour point cloud;
determining a second contour edge based on two adjacent reconstructed contour points in the plurality of reconstructed contour points;
determining a second contour point in the plurality of contour points of adjacent the first two-dimensional contour point cloud corresponding to the second contour edge; and
generating a second triangular section based on the one or more second contour points and the second contour edge.

14. The grid model generation system of claim 13, wherein the first contour point is a contour point in the second two-dimensional contour point cloud closest to the corresponding first contour edge; and
the second contour point is a common vertex to two first triangular sections respectively including the two adjacent reconstructed contour points based on which the second contour edge is determined.

15. The grid model generation system of claim 13, wherein a count of the plurality of contour points in the first two-dimensional contour point cloud is greater than a count of the plurality of contour points in the second two-dimensional contour point cloud.

16. The grid model generation system of claim 11, wherein the obtaining a three-dimensional (3D) contour point cloud related to an object includes:
obtaining a centerline of the object, wherein the centerline includes a plurality of center points;
determining a reconstruction plane at each of the plurality of center points;
generating the plurality of two-dimensional contour point clouds based on the reconstruction plane; and
generating the three-dimensional contour point cloud based on the plurality of two-dimensional contour point clouds.

17. The grid model generation system of claim 16, wherein the generating plurality of two-dimensional contour point clouds based on the reconstruction planes includes:
for each of the plurality of two-dimensional contour point clouds,
obtaining a contour expression and a contour rotation angle; and
generating the two-dimensional contour point cloud by sampling a preset count of contour points in the corresponding reconstruction plane based on the contour expression and the contour rotation angle.

18. The grid model generation system of claim 11, wherein the determining a plurality of two-dimensional (2D) contour point clouds based on the three-dimensional contour point cloud includes:
obtaining a centerline of the object based on the three-dimensional contour point cloud, wherein the centerline includes a plurality of center points;
determining a reconstruction plane at each of the plurality of center points; and
for each of the plurality of two-dimensional contour point clouds, determining the two-dimensional contour point cloud based on one or more contour points in the three-dimensional contour point cloud that have a distance from the reconstruction plane less than a threshold.

19. The grid model generation system of claim 11, wherein the grid model is configured to represent a virtual blood vessel, and the method further comprising:
selecting at least one of the plurality of two-dimensional contour point clouds; and
adjusting a contour expression of the at least one of the plurality of two-dimensional contour point clouds to simulate an abnormity of the virtual blood vessel.

20. A non-transitory computer readable medium, comprising at least one set of instructions, wherein when executed by one or more processors of a computing device, the at least one set of instructions causes the computing device to perform a method, the method comprising:
obtaining a three-dimensional (3D) contour point cloud related to an object;
determining a plurality of two-dimensional (2D) contour point clouds based on the three-dimensional contour point cloud;
generating a plurality of grid sections between two adjacent two-dimensional contour point clouds of the plurality of two-dimensional contour point clouds; and
generating a grid model of the object based on the plurality of grid sections.
